(19) United States
(12) Patent Application Publication
(10) Pub. No.: US 2006/0063219 A1
(43) Pub. Date: Mar. 23, 2006

(54) Trivalent metal mediated homogeneous luminescent proximity assay

(75) Inventors: Richard C. Kuo, Fremont, CA (US); Timothy D. Dawes, Clayton, CA (US); Timothy D. Machajewski, Martinez, CA (US)

Correspondence Address: Chiron Corporation;Intellectual Property - R440, P.O. Box 8097, Emeryville, CA, 94662-8097, US

(73) Assignee: Chiron Corporation

(21) Appl. No.: 11/230,933

(22) Filed: Sep. 19, 2005

Related U.S. Application Data
(60) Provisional application No. 60/610,799, filed on Sep. 17, 2004.

(51) Int. Cl. C12Q 1/48; C12M 1/34

(52) U.S. Cl. 435/15; 435/287.1

(57) ABSTRACT

An in vitro protein kinase assay technology that (1) exhibits a high assay signal to background ratio (S/B) and range (S-B); (2) is homogenous; (3) is non-radioactive; and (4) does not require a phospho-specific antibody involves complexing a trivalent metal ion (e.g. Ga³⁺, Fe³⁺, Al³⁺, In³⁺, Ru³⁺, Sc³⁺, Y³⁺) to the surface of amplified luminescent proximity assay acceptor or donor beads, e.g., via a suitable linker such as nitrilotriacetic acid (NTA; also referred to as carboxymethyl-lysine), iminodiacetic acid (IDA), or an appropriately substituted N-containing heterocycle, for example a triazoheterocycle, for example a triazocyclononaneononane, such as 1-propylamino-4-acetato-1,4,7-triazacyclononane. A protein (or constituent part) or other kinase substrate is bound to the surface of the other of an amplified luminescent proximity assay acceptor or donor bead and, if phosphorylated, brought into proximity with the trivalent metal ion-complexed acceptor bead to generate a luminescent signal. Presence of a kinase inhibitor inhibits phosphorylation and therefore signal generation and, in this way, is detectable. As the invention described herein recognizes the presence or absence of phosphate groups on a protein, (or constituent part), or other biological macromolecule (e.g., mono, di, or trinucleotides, cyclic nucleotides or phosphate substituted inositols), it is broadly applicable to any phosphorlylation or dephosphorylation reaction enzymes and provides a highly robust and flexible assay format for protein kinases and other enzyme classes, including lipid kinases, phosphatases, phosphodiesterases and others.

### CROSS-REFERENCE TO RELATED APPLICATIONS

This application claims priority to U.S. Provisional Patent Application No. 60/610,799, filed Sep. 17, 2004, titled TRIVALENT METAL MEDIATED HOMOGENEOUS LUMINESCENT PROXIMITY ASSAY, the disclosure of which is incorporated herein by reference in its entirety and for all purposes.

### BACKGROUND OF THE INVENTION

This invention relates to materials and methods for conducting biological assays, in particular, kinase assays.

Phosphorylation of various intracellular protein and lipid substrates plays a central role in numerous cellular processes (e.g., growth, proliferation, apoptosis, differentiation, and cell cycle progression). Kinases, the enzymes responsible for phosphorylation of cellular enzymes, including other kinases, and lipids have been identified as a major target class for potential therapeutic intervention. Kinases are broadly categorized as either tyrosine (i.e., phosphorylation occurs at tyrosine amino acid residues) or serine/threonine (i.e., phosphorylation occurs at either serine or threonine amino acid residues) kinases. Of the approximately 518 human kinases identified to date, approximately 85% are serine/threonine (S/T) kinases with the remainder classified as tyrosine kinases. Manning, G., Whyte, D. B., Martinez, R., Hunter, T. & Sudarsanam, S. The protein kinase complement of the human genome. Science 298, 1912-34 (2002).

Numerous in vitro assays have been described which allow high throughput determination of kinase activity in the presence of potential kinase inhibitory compounds. Concerns with existing high throughput screening (HTS) kinase assays, as with any HTS assay, include the cost of detection reagents and the time cost for validating and optimizing the assay. To this end, a non-radioactive assay is highly preferred as disposal of radioactive waste is very expensive. Also, homogenous assays (i.e., assays that do not require wash steps) require fewer experimental steps with a concomitant reduction in possible sources of screening artifacts and are more amenable to HTS, typically offering higher assay throughput and lower upfront and ongoing capital costs associated with equipment acquisition and maintenance.

One currently available in vitro kinase assay technology that meets these requirements is AlphaScreen™ (Amplified Luminescent Proximity Homogenous Assay) from Perkin Elmer LAS, Inc. AlphaScreen™ relies on the biomolecular interaction between conjugated donor and acceptor beads (approximately 250 nm in diameter). Ullman, E. F. et al. Luminescent oxygen channeling immunoassay: measurement of particle binding kinetics by chemiluminescence. Proc Natl Acad Sci USA 91, 5426-30 (1994). For AlphaScreen™ kinase assays, a biotinylated peptide or protein substrate is bound to the donor bead and, if phosphorylated, brought into proximity with an acceptor bead conjugated with a phospho-specific antibody (typically phospho-tyrosine specific). Warner, G., Illy, C., Pedro, L., Roby, P. & Bosse, R. AlphaScreen™ kinase HTS platforms. Curr. Med. Chem. 11, 721-730 (2004). Donor and acceptor beads which are within about 200 nm of each other emit a luminescence signal read as light emitted between 520 and 620 nm. AlphaScreen™ kinase assays routinely exhibit signal to background (S/B) ratios greater than 10 (Warner, G., Illy, C., Pedro, L., Roby, P. & Bosse, R. Alphascreen kinase HTS platforms. Curr. Med. Chem. 11, 721-730 (2004)) and can be used with large protein substrates. However, while high quality anti-phosphotyrosine antibodies are available for assay technologies that rely on phospho-specific antibodies for detection, generic anti-phosphoserine or anti-phosphothreonine antibodies are typically not of high enough quality to produce robust signals. Consequently, phospho-sequence specific antibodies are needed, which increases the time required for assay optimization and validation and add to the overall cost of the assay. The use of a phospho-specific antibody precludes use of AlphaScreen™ in screening particular S/T kinases or other enzyme classes for which antibodies of high affinity or specificity are not available.

Another currently available in vitro homogeneous, non-radioactive kinase assay technology is IMAP™ (Immobilized Metal Affinity for Phosphate) from Molecular Devices Corporation. IMAP™ utilizes nanoparticles with trivalent metal ions complexed on their surface. Sportsman, J. R., Daijo, J. & Gaudet, E. A. Fluorescence polarization assays in signal transduction discovery. Comb. Chem. High Throughput Screen 6, 195-200 (2003). It has been known for some time that trivalent transition metals (e.g., Ga³⁺, Fe³⁺, Al³⁺, In³⁺, Ru³⁺, Sc³⁺, Y³⁺) can bind phosphate groups with high affinity and specificity. Osterberg, R. Metal and hydrogen-ion binding properties of O-phosphoserine. Nature 179, 476-477 (1957); Muszynska, G., Andersson, L. & Porath, J. Selective adsorption of phosphoproteins on gel-immobilized ferric chelate. Biochemistry 25, 6850-3 (1986); and Posewitz, M. C. & Tempst, P. Immobilized gallium (III) affinity chromatography of phosphopeptides. Anal Chem. 71, 2883-2892 (1999). IMAP™ relies on the increased fluorescence polarization of a fluorescently-labeled peptide substrate when the IMAP™ nanoparticle is bound following phosphorylation. This system has the advantage of being able to bind free phosphate groups in a peptide substrate without regard to the surrounding amino acid(s) unlike phospho-antibodies. However, because fluorescence polarization measures changes in molecular mobility, in biological systems the maximum signal range (S-B) is typically limited to about 350 mP. This gives a theoretical maximum signal to background ratio (S/B) in biological assays of about 8, however in practice the S/B is often 2 or less (see, e.g., J. Biomolecular Screening 8(6): 694-700 (2003)). Moreover, in situations where the substrate is a protein or large protein fragment, the S/B becomes too small to be usable as the intrinsic polarization of a large molecule is high (i.e., B, background, increases significantly). Assays with low signal to background are more likely to generate false positives which can significantly affect the overall robustness and reproducibility of a HTS program. Walters, W. P. & Namchuk, M. Designing screens: how to make your hits a hit. Nat. Rev. Drug Dis. 2, 259-266 (2003).

Accordingly, an improved in vitro assay technology that facilitates reliable high throughput determination of kinase activity in the presence of potential kinase inhibitory compounds is needed.

### SUMMARY OF THE INVENTION

The present invention addresses these issues by providing an in vitro kinase assay technology that (1) exhibits a high assay signal to background ratio (S/B) and range (S-B); (2) is homogenous; (3) is non-radioactive; and (4) does not require a phospho-specific antibody. As the invention described herein recognizes the presence or absence of phosphate groups on a protein, (or constituent part, e.g., polypeptide or peptide), or other biological macromolecule (e.g., mono, di, or trinucleotides, cyclic nucleotides or phosphate substituted inositols), it is broadly applicable to any phosphorlylation or dephosphorylation reaction enzymes and provides a highly robust and flexible assay format for protein kinases and other enzyme classes, including lipid kinases, phosphatases, phosphodiesterases and others.

In one case, the assay involves combining amplified luminescent proximity assay donor and acceptor beads with a kinase and a kinase inhibitory compound candidate. One of the beads, generally the donor bead, has bound to its surface a biomolecule capable of phosphorylation by a kinase, such as a protein, lipid or nucleic acid (or constituent part thereof, e.g., for protein, a peptide). The other bead, generally the acceptor bead, has a trivalent metal ion complexed to its surface, e.g., via a suitable linker such as nitrilotriacetic acid (NTA; also referred to as carboxymethyl-lysine), iminodiacetic acid (IDA), or an appropriately substituted N-containing heterocycle, for example a triazoheterocycle, for example a triazocyclononaneononane, such as 1-propylamino-4-acetato-1,4,7-triazacyclononane. A chemiluminescent signal is generated when the donor and acceptor particles are in close proximity, which occurs when the kinase phosphorylates the biomolecule and the trivalent metal ion binds to the phosphate group. If the kinase inhibitory compound candidate is a kinase inhibitory compound, the chemiluminescence emitted by the assay composition relative to chemiluminescence emitted from a control composition lacking the candidate inhibitory compound is reduced or eliminated, to the extent that the kinase is inhibited. This provides an indication or a measure of kinase inhibition, or both.

Compositions and kits which may be used to conduct such as assay are also provided.

As noted above, while the invention is primarily described herein with reference to protein or constituent part (including polypeptide or peptide) kinase assays, it is broadly applicable to any phosphorlylation or dephosphorylation reaction enzymes. Alternative uses the compositions and processes of the present invention are in assays for other types of enzymes, including lipid kinases, phosphatases and phosphodiesterases.

For lipid kinases, like PI3K, the phosphorylation reaction is PI-4,5-P2 to PI-3,4,5-P. PI (phosphatidylinositol) itself can also be used as a substrate. Once PI or PIP2 is phosphorylated, the beads show an increased chemiluminescent signal.

For phosphatases and phosphodiesterases the assay would operate in the reverse of a kinase assay since phosphatases and phosphodiesterases remove phosphate groups, e.g., a phosphatase or phosphodiesterase inhibitor would generate a high chemiluminescence signal relative to controls.

These and other aspects and advantages of the present invention are described in detail below.

### BRIEF DESCRIPTION OF THE DRAWINGS

FIG. 1 illustrates components of a composition suitable for conducting a trivalent metal mediated homogeneous luminescent proximity kinase assay in accordance with the present invention.

FIGS. 2 and 3 are plots showing luminescence counts for each condition tested in Example 1.

FIGS. 4-7 are plots showing luminescence counts for each condition tested in Example 2.

### DETAILED DESCRIPTION OF THE INVENTION

Reference will now be made in detail to specific embodiments of the invention. Examples of the specific embodiments are illustrated in the accompanying drawings. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail in order not to unnecessarily obscure the present invention.

Introduction

The present invention provided an in vitro kinase assay technology that (1) exhibits a high assay signal to background ratio (S/B) and range (S-B); (2) is homogenous; (3) is non-radioactive; and (4) does not require a phospho-specific antibody. As the invention described herein recognizes the presence or absence of phosphate groups on either a protein, protein (or constituent part thereof, e.g., a polypeptide or peptide), or other biological macromolecule (e.g., mono, di, or trinucleotides, cyclic nucleotides or phosphate substituted inositols), it is broadly applicable to any phosphorlylation or dephosphorylation reaction enzymes and provides a highly robust and flexible assay format for protein kinases and other enzyme classes, including lipid kinases, phosphatases, phosphodiesterases, and others.

In one embodiment, the assay involves combining amplified luminescent proximity assay donor and acceptor beads with a kinase and a kinase inhibitory compound candidate. One of the beads, generally the donor bead, has bound to its surface a biomolecule capable of phosphorylation by a kinase, such as a protein, lipid or nucleic acid (or constituent part thereof, e.g., for protein, a peptide). The other bead, generally the acceptor bead, has a trivalent metal ion (e.g., Ga³⁺, Fe³⁺, Al³⁺, In³⁺, Ru³⁺, Sc³⁺, Y³⁺) complexed to its surface, e.g., via a suitable linker such as nitrilotriacetic acid (NTA; also referred to as carboxymethyl-lysine), iminodiacetic acid (IDA), or an appropriately substituted N-containing heterocycle, for example a triazoheterocycle, for example a triazocyclononaneononane, such as 1-propylamino-4-acetato-1,4,7-triazacyclononane. It should be understood that the surface components of the beads in this kinase assay and for any assay in accordance with the present invention can be reversed, e.g., the trivalent metal ions can be on the donor beads and the kinase substrate (biomolecule for phosphorylation/dephosphorylation) can be on the acceptor bead.

A chemiluminescent signal is generated when the donor and acceptor particles are in close proximity, which occurs when the kinase phosphorylates the biomolecule and the trivalent metal ion binds to the phosphate group. For the purposes of the present application, chemiluminescence (adj., chemiluminescent) refers to the emission of light resulting directly or indirectly from a chemical reaction of a compound (chemiluminescent compound) with a reactive chemical species including singlet oxygen, including emission of any fluorescent or phosphorescent photon. If the kinase inhibitory compound candidate is actually a kinase inhibitory compound, the chemiluminescence emitted by the assay composition relative to chemiluminescence emitted from a control composition lacking the candidate inhibitory compound is reduced or eliminated, to the extent that the kinase is inhibited. This provides an indication or a measure of kinase inhibition, or both.

Compositions and kits which may be used to conduct such as assay are also provided.

The invention will now be described with reference to one embodiment, a kinase assay, primarily with regard to a protein kinase assay.

Trivalent Metal Mediated Homogeneous Luminescent Proximity Kinase Assay

It has been known for some time that trivalent transition metal ions, including Fe³⁺, can bind phosphate groups with high affinity and specificity, and that fact has been used to advantage in kinase assays, in particular the IMAP™ technology described above. However, the use of trivalent metal ions in kinase assays other than IMAP™ is unknown. Since singlet oxygen generation at the donor bead and propagation to the acceptor bead is key to the operation of the existing amplified luminescent proximity homogeneous assay technology, as described above, the presence of singlet ion quenchers is contraindicated by the state of the art. The suppliers of the AlphaScreen™ assay technology described above explicitly note that certain transition metal ions, including Fe³⁺, have been shown to be potent singlet oxygen quenchers, and it is strongly recommended that their use be avoided in the literature provided with the AlphaScreen™ product. A Practical Guide to Working with AlphaScreen™, page 22, PerkinElmer, Inc. (2003), incorporated by reference herein in its entirety and for all purposes. Nevertheless, the present inventors have unexpectedly found that replacement of the phospho-specific antibody used in the existing amplified luminescent proximity homogeneous assay technology described above with a trivalent metal ion provides an enhanced assay.

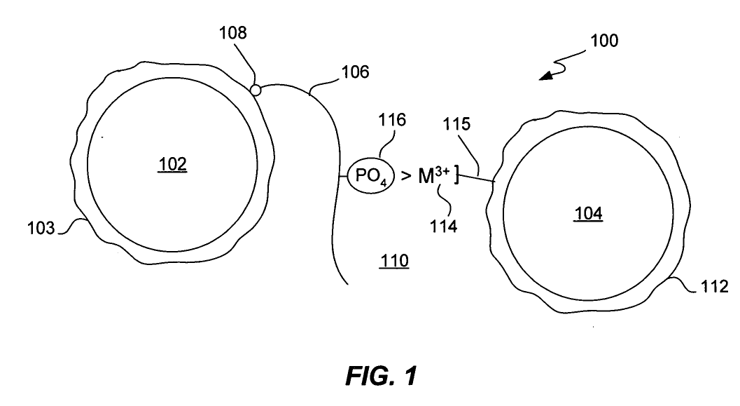

FIG. 1 illustrates components of a composition 100 suitable for conducting a trivalent metal mediated homogeneous luminescent proximity kinase assay in accordance with the present invention. The proximity assay works by making use of a biomolecular interaction between conjugated donor 102 and acceptor 104 particles. In a preferred embodiment, the particles are polymeric beads such as are described in Ullman, E. F. et al. Luminescent oxygen channeling immunoassay: measurement of particle binding kinetics by chemiluminescence. Proc Natl Acad Sci USA 91, 5426-30 (1994), and U.S. Pat. No. 6,703,248, which are incorporated by reference herein in their entirety and for all purposes. Beads in accordance with these publications are marketed as AlphaScreen™ beads, available from Perkin Elmer Life and Analytical Sciences (Boston, Mass.). The polymeric beads may be composed of one or more polymers among polystyrenes, polyacrylamides, polyvinyl chlorides, polyvinylnaphthalenes and polymethacrylates, for example. Suitable beads may be in the form of latex particles. Further, the polymeric beads include a plasticizer such as higher alkylaromatic compounds and higher alkyloxyaromatic compounds and fluorocarbons, for example in an amount of about 0.1 to about 25% by weight. The beads may be about 20 nm to 100 μm in diameter, for example about 175 nm to 275 nm in diameter. When used in a biological, e.g., kinase, assay, the beads are dispersed in an aqueous medium 110, such as a suitable buffer.

The donor bead 102 incorporates a photosensitizer that converts ambient oxygen to singlet oxygen upon excitation (activation) by a light source, for example a laser. Examples of suitable photosensitizers include endoperoxides or phthalocyanine. The donor bead also has a surface coating 103 to facilitate binding of a biomolecule 106. In general, the surface coating is complementary to a linker 108 on the biomolecule 106. For example, the donor bead surface coating 103 is an avidin, e.g., streptavidin, and the complementary linker 108 on the biomolecule 106 is biotin. Other suitable linkers include glutathione-S-transferase, protein A, protein G, and chelated Ni²⁺ for 6× histidine tagged molecules. Suitable donor beads are AlphaScreen donor beads, available from Perkin Elmer Life and Analytical Sciences (Boston, Mass.).

For kinase assays, the biomolecule 106, such as a protein, lipid or nucleic acid (or constituent part thereof, e.g., for protein, a peptide), is capable of phosphorylation by a kinase to regulate its biological activity. It is bound to the surface of the donor bead via the donor bead surface coating 103 and the linker 108. The biomolecule 106 is selected based on the kinase of interest in the particular assay. For example, if the purpose of the assay is to identify inhibitory agents to protein serine/threonine kinase PDK1, the biomolecule 106 will be a protein (or constituent part) capable of phosphorylation by PDK1, such as are known in the art. Some examples of kinases for which the assay of the present invention may be adapted are as follows: The kinase may be a protein serine/threonine kinase including the Akt kinase family, Aurora kinase family, PDK1, MAPKAP K2, Erk kinase family, CAMK kinase family, cyclin dependent kinases (e.g., CDK1, CDK2, CDK4, CDK6), RAF kinase family, casein kinase family, PKC family, PKA family, PKB family, PKG family, GSK3 beta, ROCK, SGK, Rsk family and Nek family; a protein tyrosine kinase including receptor tyrosine kinases, such as FGFR, EGFR, PDGFR, c-Kit, IGFR, insulin receptor, TrkA, TrkB, TrkC, c-Met or c-Ret, and cytoplasmic tyrosine kinases, such as Src, Lck, Lyn, Fyn, Yes, Syk, Hck, Abl and Eph family; or a lipid kinase, such as PI3 kinase, PI4 kinase or PI5 kinase.

The acceptor bead 104 incorporates a chemiluminescent compound that generates a chemiluminescent signal in the presence of singlet oxygen. Thus, when the activated donor 102 and acceptor 104 beads are in close proximity, that is, when there is no more distance between the beads than is traveled by singlet oxygen in aqueous solution during its lifetime, for example no more than about 200 nm, the chemiluminescent signal is generated. In one embodiment, the chemiluminescent compound is a thioxene derivative and emits a luminescence signal read as light emitted between 520 and 620 nm.

The acceptor particle 104 generally has a surface coating 112 to prevent non-specific binding, such as a coating of dextran or a polypeptide hydrogel. It also has a trivalent metal ion 114 complexed to the bead 104 surface, for example via a linker 115 that is covalently bonded to the acceptor particle 104 surface coating 112. The trivalent metal ion 114 may be at least one of Ga³⁺, Fe³⁺, Al³⁺, In³⁺, Ru³⁺, Sc³⁺ and Y³⁺.

Complexing a trivalent metal ion to a chromatography substrate has been shown to be possible by substituting a trivalent metal for divalent Ni²⁺ complexed nitrilotriacetic acid (NTA; also referred to as carboxymethyl-lysine) or iminodiacetic acid (IDA) resins typically used for purification of 6×His containing proteins. Porath, J. IMAC—immobilized metal ion affinity based chromatography. Trends Anal. Chem. 7, 254-259 (1988), incorporated by reference herein in its entirety and for all purposes. It has been found that a trivalent metal ion may be complexed to a polymeric bead suitable as an acceptor bead in accordance with the present invention by adaptation of this technique to the divalent Ni²⁺ complexed nitrilotriacetic acid (NTA) AlphaScreen™ acceptor beads. In addition, as described further in the examples that follow below, trivalent metals ions may be directly coupled to a resin suitable as acceptor beads, such as the AlphaScreen™ unconjugated, “raw” acceptor beads. AlphaScreen™ beads are available from Perkin Elmer Life and Analytical Sciences (Boston, Mass.).

A trivalent metal ion may also be complexed to a polymeric bead suitable as an acceptor bead in accordance with the present invention via a linker 115 that is an appropriately substituted N-containing heterocycle, such as a triazoheterocycle, for example a triazocyclononaneononane. The compound 1-acetato-4-benzyl-triazocyclononane has been described as an alternative to Ni/NTA for protein purifications and other applications (D. L. Johnson and L. L. Martin, J. Am. Chem. Soc. 2005, 127, 2018-2019 (and associated Supporting Information), incorporated herein by reference in its entirety and for all purposes). The appropriate substitutions will provide functionality to bind the triazoheterocycle to the surface of an acceptor bead 104 which, as noted above, will generally have a surface coating 112 to prevent non-specific binding, such as a coating of dextran or a polypeptide hydrogel. Appropriate substitutions include a carboxylic acid group and a free amine. One particular example of such a suitable linker 115 is 1-propylamino-4-acetato-1,4,7-triazacyclononane 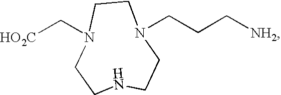
the preparation of which is described in the examples that follow below.

While not intending to be bound by theory, it is believed that a triazoheterocylic linker 115 may complex the trivalent metal ion more strongly, thereby resisting stripping of the ion from the acceptor bead by a metal ion chelator (e.g., EDTA) used as a reaction stop in an assay. Further, it is believed that the rigidity of this type of linker may also orient the complexed metal ion away from the acceptor bead surface and towards the donor bead, thereby improving signal from the assay.

As noted above, the chemiluminescent signal is generated when the activated donor and acceptor beads are held in close proximity. This can be achieved when there is a biological interaction between surface bound groups on the beads. The trivalent metal ion 114 on the acceptor bead 104 will bind to a phosphate group 116 of the biomolecule 106 to hold the beads in close proximity if the biomolecule has been phosphorylated by the kinase. If, however, the candidate compound is a kinase inhibitory compound, it will inhibit phosphorylation of the biomolecule and thus generation of the chemiluminescent signal and the chemiluminescence emitted by the assay composition relative to chemiluminescence emitted from a control composition lacking the candidate inhibitory compound is reduced or eliminated, to the extent that the kinase is inhibited. This provides an indication or a measure of kinase inhibition, or both.

Accordingly, an assay in accordance with the present invention may be conducted to detect a kinase inhibitory compound. The method involves providing a composition including a donor particle with a photosensitizer and a surface bound unphosphorylated biomolecule capable of phosphorylation by a kinase, and an acceptor particle with a trivalent metal ion complexed to the particle surface and a chemiluminescent compound. The chemiluminescent compound is characterized in that it generates a luminescent signal when the photosensitizer is activated and the donor and acceptor particles are in close proximity. The kinase and a candidate kinase inhibitory compound are introduced to the composition so that no phosphorylation occurs in the absence of the candidate compound. There are a number of ways to do this. For the phosphorylation reaction to begin, three components must be present: the kinase, the kinase substrate (e.g., a biomolecule, such a protein (or constituent part) to be phosphorylated) and ATP. Any two of the three are combined and mixed or added to wells containing the compound. The inhibitor candidate must be added before all of the necessary elements for phosphorylation to take place are combined. And then the third component is added.

It can then be determined whether the candidate compound is a kinase inhibitor or not by detecting inhibition of the kinase by the candidate inhibitory compound observed as reduced or eliminated chemiluminescence relative to chemiluminescence emitted from a control composition lacking the candidate inhibitory compound.

It should be noted that in prior luminescent proximity assays, such as those conducted with the AlphaScreen™ technology, the recommended stop buffer includes EDTA, a common and convenient metal chelator. A trivalent metal ion-mediated luminescent proximity assay in accordance with the present invention may or may not use a stop buffer that includes EDTA, depending upon the relative affinities for the trivalent metal ions of EDTA and the linker used to complex the trivalent metal ions to the acceptor bead. Where the affinity of EDTA is greater than the linker, in order to avoid the risk of stripping the trivalent metal ions from the beads when the reaction stop buffer is added, an alternative reaction stop buffer may be used in the assay. For example, a non-EDTA buffer, such as the IMAP™ binding buffer, which does not contain EDTA may be used in such instances to avoid metal ion stripping. A low pH solution can also provide an acceptable stop buffer in such instances. Where the affinity of the linker is greater than EDTA, EDTA may be used.

Accordingly, some embodiments of the present invention, for instance those using a NTA trivalent metal ion linker, do not use an EDTA-containing reaction stop buffer since EDTA is a strong enough metal chelator that it might strip the NTA complexed metal ions off the acceptor bead. One the other hand, an EDTA-containing reaction stop buffer may be used in embodiments of the present invention wherein a triazohetercyclic linker, such as 1-propylamino-4-acetato-1,4,7-triazacyclononane, is used since it is believed that such a linker complexes trivalent metal ions with greater affinity than EDTA, thereby preventing stripping the metal ions from the bead by EDTA.

### EMBODIMENTS OF THE INVENTION

The present invention may be embodied as a composition, kit or method, for example as follows:

A composition comprising a donor particle comprising a photosensitizer; an acceptor particle comprising a chemiluminescent compound, the compound characterized in that it generates a luminescent signal when the photosensitizer is activated and the donor and acceptor particles are in close proximity; and a trivalent metal ion complexed to the surface of one of the donor or acceptor particles.

A composition, comprising a polymeric particle comprising a trivalent metal ion complexed to the particle surface and one of a photosensitizer and a chemiluminescent compound. The particle may be characterized in that a luminescent signal is generated when a second particle comprising the other of the photosensitizer and the chemiluminescent compound are in close proximity and the photosensitizer is activated.

A kit for use in an assay, the kit comprising, in packaged combination: reagents for conducting an assay, the reagents comprising, a donor particle comprising a photosensitizer; an acceptor particle comprising a chemiluminescent compound, the compound characterized in that it generates a luminescent signal when the photosensitizer is activated and the donor and acceptor particles are in close proximity; and a trivalent metal ion complexed to the surface of one of the donor or acceptor particles. The kit may also contain instructions for conducting a trivalent metal mediated homogeneous luminescent proximity assay, such as a method of detecting a kinase inhibitory compound according to that aspect of the invention described below.

A method of detecting a kinase inhibitory compound, the method comprising: providing a composition comprising, a donor particle comprising a photosensitizer and a surface bound unphosphorylated biomolecule capable of phosphorylation by a kinase, and an acceptor particle comprising a trivalent metal ion complexed to the particle surface and a chemiluminescent compound, the compound characterized in that it generates a luminescent signal when the photosensitizer is activated and the donor and acceptor particles are in close proximity; introducing to the composition a candidate kinase inhibitory compound; introducing to the composition the kinase; and determining that the candidate kinase inhibitory compound is a kinase inhibitory compound by detecting inhibition of the kinase by the candidate inhibitory compound observed as reduced chemiluminescence relative to chemiluminescence emitted from a control composition lacking the candidate inhibitory compound.

A method of detecting a phosphatase or phosphodiesterase inhibitory compound, the method comprising: providing a composition comprising, a donor particle comprising a photosensitizer and a surface bound phosphorylated biomolecule capable of dephosphorylation by a phosphatase or phosphodiesterase, and an acceptor particle comprising a trivalent metal ion complexed to the particle surface and a chemiluminescent compound, the compound characterized in that it generates a luminescent signal when the photosensitizer is activated and the donor and acceptor particles are in close proximity; introducing to the composition the phosphatase or phosphodiesterase; introducing to the composition a candidate phosphatase or phosphodiesterase inhibitory compound; and determining that the candidate phosphatase or phosphodiesterase inhibitory compound is a phosphatase or phosphodiesterase inhibitory compound by detecting inhibition of the phosphatase or phosphodiesterase by the candidate inhibitory compound observed as increased chemiluminescence relative to chemiluminescence emitted from a control composition lacking the candidate inhibitory compound.

For any of the compositions, kits or methods of the invention: The activated photosensitizer may convert ambient oxygen to singlet oxygen upon excitation by a light source. The light source may be a laser. The photosensitizer may be an endoperoxide. The chemiluminescent compound may be a thioxene derivative. The acceptor and donor particles may be dispersed in an aqueous medium. The close proximity is no more than the distance traveled by singlet oxygen in aqueous solution during its lifetime, e.g., no more than 200 nm. The particles may be polymeric beads. The polymeric beads may comprise a polymer selected from the group consisting of polystyrenes, polyacrylamides, polyvinyl chlorides, polyvinylnaphthalenes and polymethacrylates. The polymeric beads may comprise about 0.1 to about 25% by weight of a plasticizer selected from the group consisting of higher alkylaromatic compounds and higher alkyloxyaromatic compounds and fluorocarbons. In some cases, the polymeric beads may be about 20 nm to about 100 μm in diameter. In other cases, the polymeric beads may be about 175 nm to about 275 nm in diameter. The beads may alternatively comprise latex particles. In some cases, the trivalent metal ion may be complexed to the acceptor particle surface; in others, to the donor particle surface. The trivalent metal ion may be complexed to the surface of one of the donor or acceptor particles via a N-containing linker, such as one of carboxymethyl-lysine and a 1-propylamino-4-acetato-1,4,7-triazacyclononane linker, e.g, a 1-propylamino-4-acetato-1,4,7-triazacyclononane linker. The donor particle may further comprise a surface coating to facilitate binding of a biomolecule capable of phosphorylation by a kinase. The coating may comprise one of a complementary binding pair. The donor bead may further comprise a surface bound biomolecule selected from the group consisting of proteins, lipids, nucleic acids and their constituent parts, the biomolecule capable of phosphorylation by a kinase to regulate biological activity of the biomolecule. The kinase may be selected from the group consisting of protein serine/threonine kinases, protein tyrosine kinases and lipid kinases. For example, the kinase may be a protein serine/threonine kinase selected from the group consisting of Akt kinase family, Aurora kinase family, PDK1, MAPKAP K2, Erk kinase family, CAMK kinase family, cyclin dependent kinase, RAF kinase family, casein kinase family, PKC family, PKA family, PKB family, PKG family, GSK3 beta, ROCK, SGK, Rsk family and Nek family; or the kinase may be a protein tyrosine kinase selected from the group consisting of receptor tyrosine kinases and cytoplasmic tyrosine kinases; or the kinase may be a receptor tyrosine kinase selected from the group consisting of FGFR, EGFR, PDGFR, c-Kit, IGFR, insulin receptor, TrkA, TrkB, TrkC, c-Met and c-Ret; or the kinase may be a cytoplasmic tyrosine kinase selected from the group consisting of Src, Lck, Lyn, Fyn, Yes, Syk, Hck, Abl and Eph family; or the kinase may be a lipid kinase selected from the group consisting of PI3 kinase, PI4 kinase and PI5 kinase. The biomolecule may further comprise a linker complementary to the surface coating on the donor bead. The donor bead coating may comprise an avidin, e.g., streptavidin, and the complementary linker on the biomolecule may comprise biotin. A biological interaction between the trivalent metal ion on the acceptor bead and a phosphate group of the phosphorylated biomolecule on the donor bead may hold the beads in close proximity. The trivalent metal ion may be selected from the group consisting of Ga³⁺, Fe³⁺, Al³⁺, In³⁺, Ru³⁺, Sc³⁺ and Y³⁺. In the methods of the invention, the candidate kinase inhibitor compound may be introduced prior to or simultaneously with the introduction of the kinase, and ATP may be further introduced to the composition.

### EXAMPLES

The following examples are provided to illustrate certain aspects of the present invention and component materials and their preparation. The examples will serve to further illustrate the invention but are not meant to limit the scope of the invention in any way.

### Example 1

Materials and Methods

AlphaScreen™ streptavidin donor beads, AlphaScreen™ Ni²⁺-chelate acceptor beads and AlphaScreen™ unconjugated, “raw” acceptor beads were purchased from Perkin Elmer Life and Analytical Sciences (Boston, Mass.). IMAP™ binding buffer was obtained from Molecular Devices (Sunnyvale, Calif.) as a 5× concentrate. Trivalent metal ion salts were purchased from Sigma Aldrich, St. Louis, Mo. (FeCl₃) and Alfa Aesar, Ward Hill, Mass. (GaCl₃ and AlCl₃). Akt3 kinase and biotinylated Crosstide substrate were purchased from Upstate, Charlottesville, Va. Sodium cyanoborohydride and carboxymethyl-L-lysine were purchased from Sigma Aldrich. Other solutions described herein were made from research grade material obtained from VWR International, West Chester, Pa. Initially, two methods of complexing trivalent metal ions to AlphaScreen™ acceptor beads were developed and are described below.

Method 1: Substitution of Trivalent Metal Ion for Ni²⁺ on Ni²⁺-Chelate AlphaScreen™ Beads
1) Pellet 250 μg (50 μl of 5 mg/ml) Ni²⁺-chelate acceptor beads by centrifugation (13,000×g for 20 min at 4° C.).
2) Re-suspend pelleted beads in 1 mL strip buffer (500 mM NaCl, 100 mM EDTA, 50 mM Tris HCl, pH 8). Vortex, sonicate and incubate at 37° C. for 1 hr. in dark.
3) Wash 3× (13,000×g for 20 min at 4° C.) in 0.1 M Tris HCl, pH 8.
4) Re-suspend in 1 mL of 100 mM M³⁺ solution (e.g., FeCl₃, GaCl₃, or GaNO₃). Vortex, sonicate and incubate at 37° C. for 1 hr. in dark.
5) Wash 2× in 1 mL storage buffer (25 mM HEPES/100 mM NaCl, pH 7.4).
6) Re-suspend final wash in 100 μl storage buffer.

Method 2: Direct Coupling of Trivalent Metal Ion to AlphaScreen™ Acceptor Beads Via NTA (carboxymethyl-lysine)
1) Combine the following in a 1.5 ml microfuge tube:
   400 μg of raw AlphaScreen™ aldehyde acceptor beads (40 μl of 20 mg/ml)
   10 μl of 1% Tween-20
   8 μl of NaCNBH₄ (sodium cyanoborohydride) solution (25 mg/ml)
   400 μg of carboxymethyl-lysine (40 μl of 20 mg/ml in 0.1 M MOPS, pH 8)
   62 μl of 0.1 M MOP, pH 8
2) Incubate reaction with shaking for 48 to 60 hrs. at 37° C. in dark.
3) Block reaction by adding 10 μl of 1 M Tris-HCl, pH 7.5. Incubate for 1 hr. at 37° C. in dark.
4) Pellet beads, decant supernatant and re-suspend with vortexing in 200 μl of 0.1M Tris-HCl, pH 8.
5) Repeat step 4 twice.
6) Re-suspend beads in 100 mM M³⁺ solution (e.g., FeCl₃, GaCl₃, or GaNO₃) prepared in 1.5 mM NaOH. Incubate for 1 hr. at 37° C. in dark.
7) Wash by pelleting at 13,000×g for 20 min. at 4° C. and re-suspend in 200 μl of 0.1 M Tris-HCl, pH 8.
8) Repeat step 7 twice.
9) After final wash, re-suspend pellet in acceptor bead storage buffer (25 mM HEPES/100 mM NaCl, pH 7.4).

Results

Method 1:

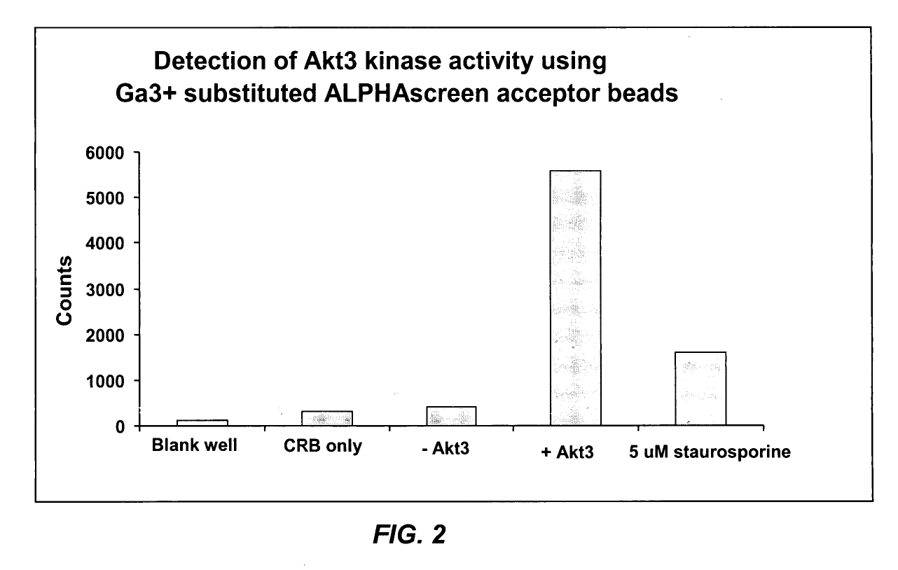

Protocol. Biotinylated Crosstide peptide (552 μM stock solution) and ATP (10 mM stock solution) were diluted to 1 μM and 20 μM, respectively, in complete reaction buffer (CRB: 10 mM Tris HCl, 10 mM MgCl₂, 0.01% BSA, 1 mM DTT, pH 7.2) to make a 2× concentrated substrate/ATP solution. Akt3 kinase (Upstate, approximately 2 μM stock solution) was diluted to 20 nM in CRB to make a 2× concentrated kinase solution. 10 μl of the 2× substrate/ATP solution was combined with either 10 ul of CRB alone or 10 ul of 2×Akt3 kinase solution (final assay concentrations: 10 nM Akt3, 10 uM ATP, 500 nM Crosstide) and incubated for 2 hr at room temperature. As a control some reactions were pre-incubated for 15 min with 5 uM staurosporine, a potent pan-kinase inhibitor. The reaction was stopped by adding 30 ul of a mixture of AlphaScreen™ streptavidin donor (10 ug/ml) and trivalent metal ion complexed acceptor (20 ug/ml) beads in 3× concentrated IMAP™ binding buffer. Luminescence counts for each condition were read on a Packard Fusion Alpha and shown in FIGS. 1 and 2 (values shown in FIGS. 1 and 2 are the average of 4 replicates).

Method 2:

Protocol. Biotinylated Crosstide peptide (552 uM stock solution) and ATP (10 mM stock solution) was diluted to 1 uM in complete reaction buffer (CRB: 10 mM Tris HCl, 10 mM MgCl₂, 0.01% BSA, 1 mM DTT, pH 7.2) to make a 2× concentrated substrate/ATP solution. This solution was subsequently serially diluted 1:1 to create solutions of fixed peptide concentration (1 uM), but varying ATP concentrations (20, 10, 5, and 2.5 uM). Akt3 kinase (Upstate, approximately 2 uM stock solution) was serially diluted in CRB to make a 2× concentrated kinase solution (20, 10, 5, and 2.5 nM).

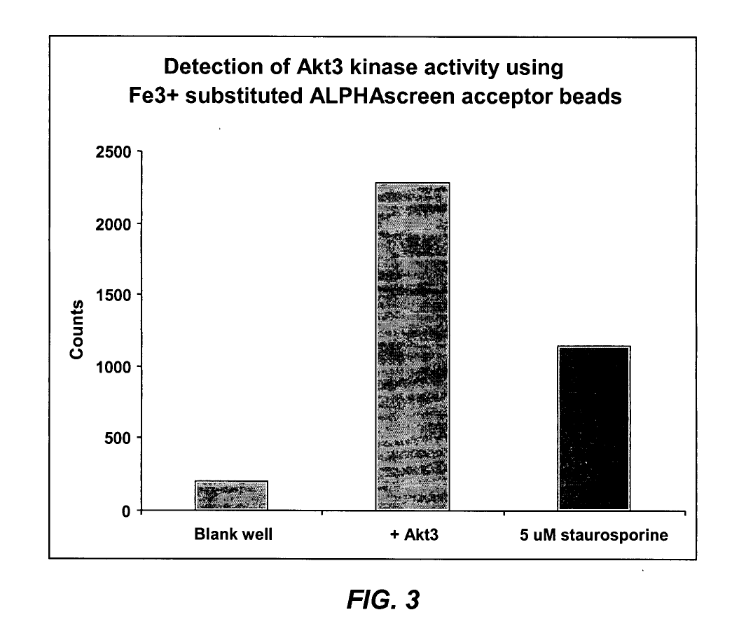

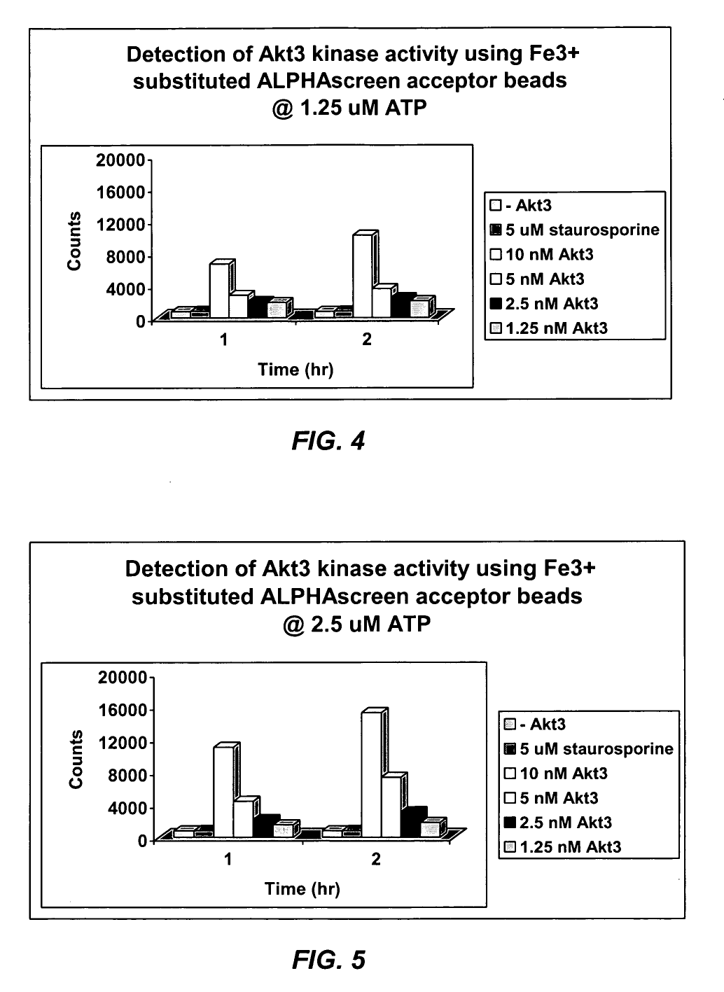

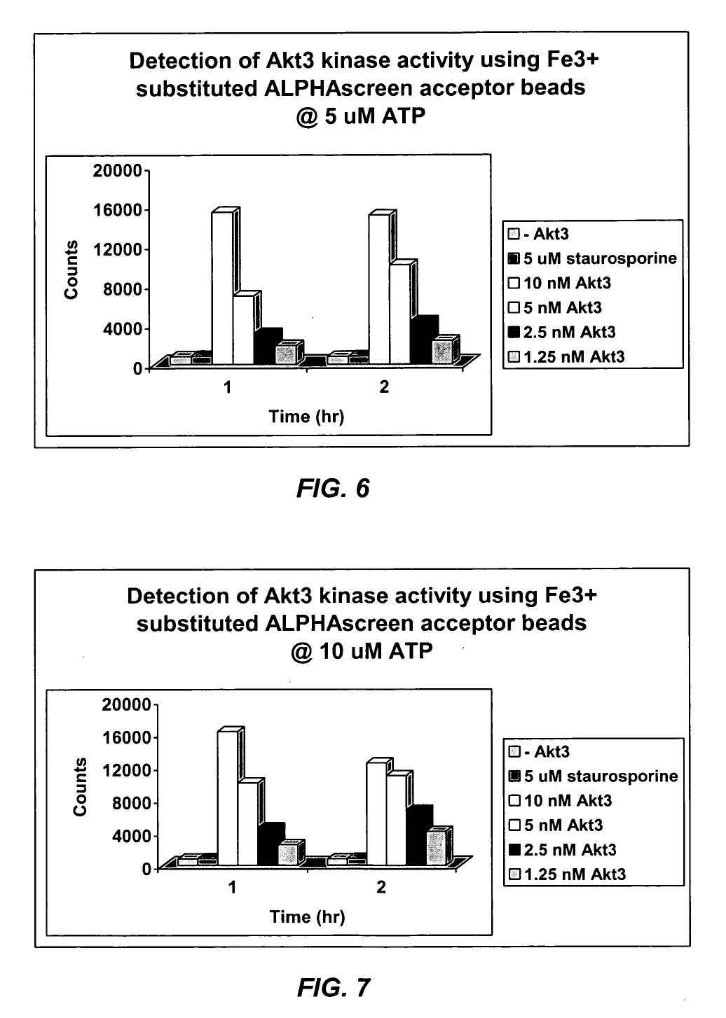

10 ul of each of the 2× substrate/ATP solutions was combined with either 10 ul of CRB alone or 10 ul of each of 2×Akt3 kinase solution concentrations (final assay concentrations: 1.25 to 10 nM Akt3, 1.25 to 10 uM ATP, 500 nM Crosstide) and incubated for 1 and 2 hr at room temperature. As a control some reactions were pre-incubated for 15 min with 5 uM staurosporine. The reaction was stopped by first adding 15 ul of a mixture of AlphaScreen™ streptavidin donor (0.1 ug/ul) followed by 15 ul Fe³⁺ complexed acceptor (0.2 ug/ul) beads in 3× concentrated IMAP™ binding buffer after the final 2 hr. incubation period. Luminescence counts for each condition were read on a Packard Fusion Alpha and shown in FIGS. 3-6.

Discussion

The results clearly indicate that trivalent metal ion (M³⁺) substituted AlphaScreen™ acceptor beads can be a powerful and simple method for rapidly optimizing and validating a kinase assay. M³⁺ substituted beads were readily made and successfully detected Crosstide phosphorylation in initial experiments with a S/B ratio of about 20.

### Example 2

### Direct coupling of trivalent metal ion to AlphaScreen™ acceptor beads via 1-propylamino-4-acetato-1,4,7-triazacyclononane

Trivalent metal ions may be coupled to AlphaScreen™ acceptor beads via 1-propylamino-4-acetato-1,4,7-triazacyclononane or another N-containing heterocycle in the same manner as described for coupling via NTA in Method 2, above. Briefly, raw AlphaScreen™ aldehyde acceptor beads may be combined with buffered 1-propylamino-4-acetato-1,4,7-triazacyclononane, Tween-20 and NaCNBH₄ (sodium cyanoborohydride) solution. The reaction is incubated with shaking for 48 to 60 hrs. at 37° C. in the dark, then blocked by adding 1 M Tris-HCl, pH 7.5, before being incubated again for 1 hr. at 37° C. in the dark. The beads are then pelleted, the supernatant decanted, and the pelleted beads re-suspended in 0.1M Tris-HCl, pH 8. This pelleting and resuspension process is repeated twice before the beads are re-suspended in 100 mM M³⁺ solution (e.g., FeCl₃, GaCl₃, or GaNO₃) prepared in 1.5 mM NaOH and incubated for 1 hr. at 37° C. in the dark. The beads are then washed by pelletings and re-suspension in 0.1 M Tris-HCl, pH 8 several times before finally being re-suspended in acceptor bead storage buffer (25 mM HEPES/100 mM NaCl, pH 7.4).

### Example 3

### Synthesis of 1-propylamino-4-acetato-1,4,7-triazacyclononane

1-propylamino-4-acetato-1,4,7-triazacyclononane may be obtained or prepared by any suitable technique, and has, for example, been synthesized according to the following modification of the procedure found in A. Warden, B. Graham, M. T. W. Hearn, L. Spiccia Org. Letters, 2001, 3, 2855-2858 (and Associated Supporting Information), incorporated herein by reference in its entirety and for all purposes.

1,4,7,-Triazacyclononane tris HCl (2.0 g) was dissolved in water (12 mL). Sodium hydroxide (1.3 g) was added portion-wise over 30 min and the resulting solution was stirred at room temperature for 1 h. The mixture was concentrated in vacuo to a white paste, which was further dried by concentration from toluene several times. Toluene was added to the resulting residue and the resulting suspension was sonicated for several minutes then decanted to remove the white solid. Dimethylformamide dimethyl acetate was added to the toluene solution and the mixture was heated at reflux for 4 h. The reaction mixture was cooled to room temperature and concentrated to afford a slightly yellow oil. This oil was taken up in acetoniltrile (5 mL) and N-(3-bromopropyl)phthalimide (2.1 g) was added resulting in a deep yellow solution. The solution was stirred overnight resulting in a white precipitate, which was collected by filtration and washed with acetonitrile and ether (additional solid was recovered from the filtrate) to afford 2.2 g (67% of theoretical) of 1-(N-(3-propyl)phthalimido)-1,4,7-triazacyclononane orthoamidinium bromide. LC/MS Rt=1.4 min, m/z=327.

1-(N-(3-propyl)phthalimido)-1,4,7-triazacyclononane orthoamidinium bromide (870 mg) was dissolved in water (10 mL) and heated at reflux overnight then concentrated in vacuo. Residual water was removed by co-evaporation with toluene resulting in a viscous yellow oil, which was immediately taken up in acetonitrile. After sonication for 5 min, sodium carbonate (2 g) and ethyl bromoacetate (0.29 mL) was added and the resulting mixture was heated at reflux for 2 days. After cooling to room temperature, the solids were removed by filtration and the filtrate was concentrated in vacuo to a yellow oil. The oil was taken up in water and extracted into chloroform. The combined organics were dried over sodium sulfate, filtered and concentrated to afford 499 mg of a slightly yellow oil. This oil was dissolved in 5 M HCl (11 mL) and heated at 93° C. for 20 h. The solution was cooled to room temperature then placed in the refrigerator overnight resulting in the formation of a white precipitate. The solid was removed by filtration and the filtrate was concentrated to an oily residue, which was dissolved in hydrobromic acid (4 mL) and acetic acid (4 mL). Ether was added until two layers were observed and the mixture was stored in the refrigerator for 3 days. The resulting white solid was collected by filtration under a blanket of nitrogen and washed with water. The solid was further dried in vacuo to afford 300 mg of the title compound. LC/MS Rt=0.3 min m/z=245. NMR (1H, 300 MHz) identical to published data.

### Alternative Embodiments

As noted above, while the invention is primarily described herein with reference to protein kinase assays, it is broadly applicable to any phosphorlylation or dephosphorylation reaction enzymes. Alternative uses the compositions and processes of the present invention are in assays for other types of enzymes, including lipid kinases, phosphatases and phosphodiesterases.

For lipid kinases, like PI3K, the physiological reaction is PI-4,5-P2 to PI-3,4,5-P. PI (phosphatidylinositol) itself can also be used as a substrate. Once PI or PIP2 is phosphorylated, the beads show an increased signal due to the addition of phosphate.

For phosphatases and phosphodiesterases the assay would operate in the reverse of a kinase assay since phosphatases and phosphodiesterases remove phosphate groups, e.g., a phosphatase or phosphodiesterase inhibitor would generate a high chemiluminescence signal relative to controls.

### CONCLUSION

While this invention has been described in terms of a few preferred embodiments, it should not be limited to the specifics presented above. Many variations on the above-described preferred embodiments, may be employed. Therefore, the invention should be broadly interpreted with reference to the following claims.

All references cited herein are incorporated by reference in their entirety and for all purposes.

What is claimed is:

1. A composition comprising:
a donor particle comprising a photosensitizer;
an acceptor particle comprising a chemiluminescent compound, the compound characterized in that it generates a luminescent signal when the photosensitizer is activated and the donor and acceptor particles are in close proximity; and
a trivalent metal ion complexed to the surface of one of the donor or acceptor particles.

2. The composition of claim 1, wherein the activated photosensitizer converts ambient oxygen to singlet oxygen upon excitation by a light source.

3. The composition of claim 2, wherein the light source is a laser.

4. The composition of claim 3, wherein the photosensitizer is an endoperoxide.

5. The composition of claim 2, wherein the chemiluminescent compound is a thioxene derivative.

6. The composition of claim 1, wherein said particles are dispersed in an aqueous medium.

7. The composition of claim 6, wherein the close proximity is no more than the distance traveled by singlet oxygen in aqueous solution during its lifetime.

8. The composition of claim 7, wherein the distance is no more than 200 nm.

9. The composition of claim 1, wherein the particles are polymeric beads.

10. The composition of claim 1, wherein the trivalent metal ion is complexed to the acceptor particle surface.

11. The composition of claim 1, wherein the trivalent metal ion is complexed to the donor particle surface.

12. The composition of claim 1, wherein the trivalent metal ion is complexed to the surface of one of the donor or acceptor particles via a N-containing linker.

13. The composition of claim 12, wherein the trivalent metal ion is complexed to the surface of one of the donor or acceptor particles via one of carboxymethyl-lysine and a 1-propylamino-4-acetato-1,4,7-triazacyclononane linker.

14. The composition of claim 1, wherein the trivalent metal ion is selected from the group consisting of Ga³⁺, Fe³⁺, Al³⁺, In³⁺, Ru³⁺, Sc³⁺ and Y³⁺.

15. A composition comprising:
a donor particle comprising a photosensitizer;
an acceptor particle comprising a chemiluminescent compound, the compound characterized in that it generates a luminescent signal when the photosensitizer is activated and the donor and acceptor particles are in close proximity; and
a trivalent metal ion complexed to the surface of the acceptor particle via a 1-propylamino-4-acetato-1,4,7-triazacyclononane linker.

16. A kit for use in an assay, said kit comprising, in packaged combination:
reagents for conducting an assay, said reagents comprising,
a donor particle comprising a photosensitizer;
an acceptor particle comprising a chemiluminescent compound, the compound characterized in that it generates a luminescent signal when the photosensitizer is activated and the donor and acceptor particles are in close proximity; and
a trivalent metal ion complexed to the surface of one of the donor or acceptor particles.

17. The kit of claim 16, wherein the trivalent metal ion is complexed to the surface of one of the donor or acceptor particles via a N-containing linker.

18. The kit of claim 17, wherein the trivalent metal ion is complexed to the surface of one of the donor or acceptor particles via one of carboxymethyl-lysine and a 1-propylamino-4-acetato-1,4,7-triazacyclononane linker.

19. The kit of claim 18, wherein the trivalent metal ion is complexed to the surface of the acceptor particle via a 1-propylamino-4-acetato-1,4,7-triazacyclononane linker.

20. The kit of claim 16, further comprising instructions for conducting a trivalent metal mediated homogeneous luminescent proximity assay.

21. A method of detecting a kinase inhibitory compound, the method comprising:
providing a composition comprising,
a donor particle comprising a photosensitizer and a surface bound unphosphorylated biomolecule capable of phosphorylation by a kinase, and
an acceptor particle comprising a trivalent metal ion complexed to the particle surface and a chemiluminescent compound, the compound characterized in that it generates a luminescent signal when the photosensitizer is activated and the donor and acceptor particles are in close proximity;
introducing to the composition a candidate kinase inhibitory compound;
introducing to the composition the kinase; and
determining that the candidate kinase inhibitory compound is a kinase inhibitory compound by detecting inhibition of the kinase by the candidate inhibitory compound observed as reduced chemiluminescence relative to chemiluminescence emitted from a control composition lacking the candidate inhibitory compound.

22. The method of claim 21, wherein the trivalent metal ion is complexed to the surface of the acceptor particle via a N-containing linker.

23. The method of claim 22, wherein the trivalent metal ion is complexed to the surface of the acceptor particle via one of carboxymethyl-lysine and a 1-propylamino-4-acetato-1,4,7-triazacyclononane linker.

24. The method of claim 23, wherein the trivalent metal ion is complexed to the surface of the acceptor particle via a 1-propylamino-4-acetato-1,4,7-triazacyclononane linker.

25. The method of claim 21, wherein the candidate kinase inhibitor compound is introduced prior to introduction of the kinase.

26. The method of claim 21, wherein the candidate kinase inhibitor compound and the kinase and introduced simultaneously.

27. A method of detecting a phosphatase or phosphodiesterase inhibitory compound, the method comprising:
providing a composition comprising,
a donor particle comprising a photosensitizer and a surface bound phosphorylated biomolecule capable of dephosphorylation by a phosphatase or phosphodiesterase, and
an acceptor particle comprising a trivalent metal ion complexed to the particle surface and a chemiluminescent compound, the compound characterized in that it generates a luminescent signal when the photosensitizer is activated and the donor and acceptor particles are in close proximity;
introducing to the composition the phosphatase or phosphodiesterase;
introducing to the composition a candidate phosphatase or phosphodiesterase inhibitory compound; and
determining that the candidate phosphatase or phosphodiesterase inhibitory compound is a phosphatase or phosphodiesterase inhibitory compound by detecting inhibition of the phosphatase or phosphodiesterase by the candidate inhibitory compound observed as increased chemiluminescence relative to chemiluminescence emitted from a control composition lacking the candidate inhibitory compound.
